(19) United States
(12) Patent Application Publication
(10) Pub. No.: US 2024/0124438 A1
(43) Pub. Date: Apr. 18, 2024

(54) PHENANTHROLINE-BASED COMPOUND, METHOD OF PREPARING THE SAME AND PEROVSKITE SOLAR CELL COMPRISING THE SAME

(71) Applicant: GWANGJU INSTITUTE OF SCIENCE AND TECHNOLOGY, Gwangju (KR)

(72) Inventors: Kwang Hee LEE, Gwangju (KR); Hee Joo KIM, Gwangju (KR); Yong Ryun KIM, Gwangju (KR); Ju Hyeon KIM, Gwangju (KR)

(21) Appl. No.: 18/239,728

(22) Filed: Aug. 29, 2023

(30) Foreign Application Priority Data
Oct. 4, 2022 (KR) 10-2022-0126245

(51) Int. Cl. C07D 471/04; H10K 30/40

(52) U.S. Cl. CPC C07D 471/04; H10K 30/40

(57) ABSTRACT

Provided are a phenanthroline-based compound, a method of preparing the same, and a perovskite solar cell including the same. The phenanthroline-based compound may be formed as a uniform layer even by a solution process due to its low average surface roughness (root mean square, RMS) and excellent processability. When a layer having the phenanthroline-based compound is provided as a polymer functional layer of the perovskite solar cell, the ionic defects on the surface between the perovskite and a metal oxide, may be passivated and charge transfer may be facilitated so that the energy efficiency and photostability of the solar cell are improved.

### CLAIM FOR PRIORITY

This application claims priority to Korean Patent Application No. 2022-0126245 filed on Oct. 4, 2022 in the Korean Intellectual Property Office (KIPO), the entire contents of which are hereby incorporated by reference.

### BACKGROUND

### 1. Technical Field

Embodiments of the present inventive concept disclose a novel phenanthroline-based compound aimed at improving the photostability and efficiency of solar cells. The compound exhibits the unique ability to passivate defects within the crystal structure of the perovskite photoactive layer, facilitating efficient charge transfer in the charge transport layer. Additionally, the invention encompasses a method for preparing this phenanthroline-based compound and its integration into a high-performance perovskite solar cell.

### 2. Related Art

Research on renewable and sustainable energy sources is being actively conducted due to carbon dioxide emission regulations, the depletion of fossil fuels, and the sudden rise of oil prices, caused by global environmental problems. Among these, solar cells, which directly convert solar energy into electrical energy, have gained significant attention. Solar cells utilize the photovoltaic effect, in which light energy from sunlight is absorbed, generating electrons and holes to produce an electrical current in the cell. They have drawn considerable interest due to their pollution-free nature, as they do not produce atmospheric pollution, noise, or vibrations.

Organic-inorganic hybrid perovskite materials possess excellent electrical and optical properties and processability in solution form. These characteristics enable the fabrication of perovskite solar cells on various substrates, including flexible ones, such as glass, which offers significant advantages of low-cost production compared to high efficiency. Due to these benefits, perovskite materials have gained considerable attention as promising candidates for next-generation solar cells.

In detail, a solar cell using an organic-inorganic hybrid perovskite broadly consists of anode and cathode electrodes, n-type and p-type charge transport layers, and a perovskite photoactive layer. Generally, there are ionic defects since an organic-inorganic hybrid perovskite material is crystallized through a thermal treatment process after a thin film is formed using a solution process. Particularly, the roughness and chemical properties of the underlying charge transport layer may affect the growth direction of perovskite grains, and the wrong growth direction of these grains and the defects of a semiconductor material degrade the performance of the perovskite solar cell. Accordingly, as disclosed in Related Art 1 (Korean Patent No. 10-1811243), various studies are being conducted to solve ionic defects.

Additionally, the hole transport layer responsible for transporting the generated holes within the perovskite photoactive layer is susceptible to moisture, leading to a decrease in the overall performance of the solar cell. This hygroscopic nature of hole transport layer affects the degradation of the perovskite material.

Therefore, there is a demand for technological advancements that can suppress the inherent defects in each semiconductor material of perovskite solar cells while minimizing the loss of charge transport during solar cell operation. The development of such technology is crucial to mitigate the performance degradation observed in perovskite solar cells.

### RELATED ART DOCUMENT

### Patent Document

Korean Patent No. 10-1811243

### SUMMARY

To solve the above-described problems, example embodiments of the present inventive concept provide a phenanthroline-based compound which is able to inhibit ionic defects in a semiconductor material, a method of preparing the same, and a perovskite solar cell including the same.

In addition, example embodiments of the present inventive concept provide a phenanthroline-based compound which is able to smoothly induce charge transfer in a solar cell, a method of preparing the same, and a perovskite solar cell including the same.

In some example embodiments, a phenanthroline-based compound represented by Formula 1 below is provided.

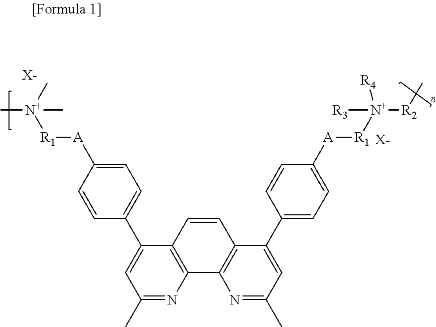

(In Formula 1, n is an integer of 1 to 1,000, R₁, R₂, R₃, and R₄ are C₁ to C₃₀ alkyls, A is oxygen, nitrogen, or a C₁ to C₁₀ alkyl, and X is a halogen.)

In Formula 1, each of Ri and R₂ is hexyl, R₃ and R₄ are each independently substituted or unsubstituted methyl, A is oxygen (O), and X is bromine (Br).

In other example embodiments, a method of preparing the above-described phenanthroline-based compound, which includes forming a first mixture by mixing a phenanthroline compound of Formula 2 below with 1,6-dibromo hexane and 2,2,2-trifluoroethanol and stirring the resulting mixture, and forming a compound of

Formula 3 below by adding sodium carbonate to the first mixture and heating the resulting mixture is provided.

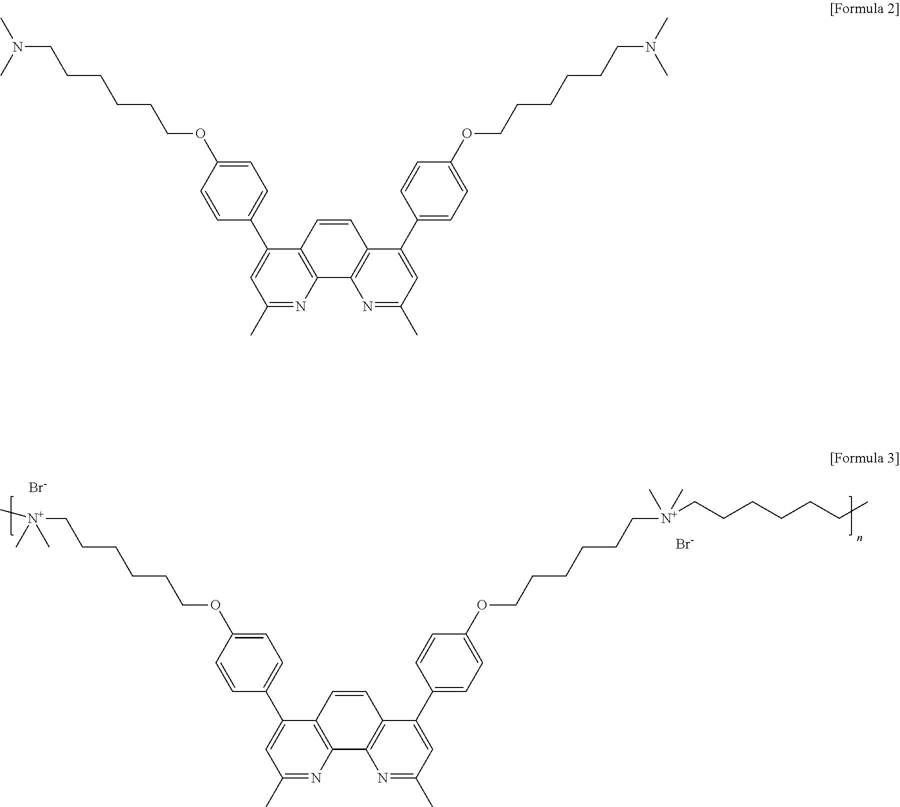

In the forming of the compound of Formula 3, heating is performed at 70° C. to 80° C.

The compound of Formula 2 is formed by a process of forming a first mixture by mixing 4,7-bis [4-(6-bromohexyl)oxy)phenyl]-2,9-dimethyl-1,10-phenanthroline with tetrahydrofuran, a process of forming a second mixture by mixing dimethylamine with the first mixture, a process of forming a third mixture by sequentially adding sodium hydroxide, ethyl acetate, and water to the second mixture, and a process of performing vacuum distillation by washing the third mixture with water.

The first mixture is formed at −80° C. to −70° C.

In still other example embodiments, a perovskite solar cell, which includes a bottom electrode, a charge transport layer formed on the bottom electrode, a polymer functional layer formed on the charge transport layer and including the phenanthroline-based compound of Formula 1, a perovskite photoactive layer formed on the polymer functional layer, a hole transport layer formed on the perovskite photoactive layer, and a top electrode formed on the hole transport layer, is provided.

The polymer functional layer has a passivation function.

The polymer functional layer is formed to a thickness of 5 nm to 14 nm.

The polymer functional layer has an average surface roughness (root mean square, RMS) value of 0.18 nm to 0.2 nm.

In terms of photostability, the aforementioned perovskite solar cell exhibits a remarkable characteristic. When exposed to light and measured using the maximum power point tracking method in a nitrogen atmosphere, the solar cell maintains approximately 93% of its initial efficiency even after 750 hours of continuous exposure. This exceptional photostability ensures that the performance degradation over time is significantly minimized, contributing to the long-term efficiency and reliability of the perovskite solar cell.

### BRIEF DESCRIPTION OF DRAWINGS

Example embodiments of the present inventive concept will become more apparent by describing in detail example embodiments of the present inventive concept with reference to the accompanying drawings, in which:

FIG. 1 is a flowchart showing a method of preparing a phenanthroline-based compound according to one embodiment of the present inventive concept;

FIG. 2 is a schematic diagram illustrating a perovskite solar cell according to one embodiment of the present inventive concept;

FIG. 3 is an image illustrating the cross-section of a perovskite solar cell according to one embodiment of the present inventive concept;

FIG. 4 is a set of images and a graph, which show the result of RMS measurement on the surfaces of a film consisting of a phenanthroline-based compound of Preparation Example 1 of the present inventive concept and a film consisting of general-purpose bathocuproine (BCP);

FIG. 5 is a set of images of measuring 2D grazing-incidence X-ray diffraction (GIXD) of perovskite photoactive layers in perovskite solar cells according to Preparation Example 2 and Comparative Example 1 of the present inventive concept;

FIG. 6 is a set of graphs illustrating 1-D(one-dimension) extraction results for perovskite photoactive layers in perovskite solar cells according to Preparation Example 2 and Comparative Example 1 of the present inventive concept;

FIG. 7 is an azimuthal diagram of perovskite photoactive layers in perovskite solar cells according to Preparation Example 2 and Comparative Example 1 of the present inventive concept;

FIG. 8 is a set of images observing charge transport layers in perovskite solar cells according to Preparation Example 2 and Comparative Example 1 of the present inventive concept using a Kelvin probe force microscope;

FIG. 9 is a set of graphs illustrating the result of measuring 1-D extracted from the images observing the charge transport layers in perovskite solar cells according to Preparation Example 2 and Comparative Example 1 of the present inventive concept using a Kelvin probe force microscope;

FIG. 10 is a graph for oxygen elements in X-ray photoelectron spectroscopic data of the charge transport layers in perovskite solar cells according to Preparation Example 2 and Comparative Example 1 of the present inventive concept;

FIG. 11 is a set of graphs illustrating space charge limited current curves for the charge transport layers in perovskite solar cells according to Preparation Example 2 and Comparative Example 1 of the present inventive concept;

FIG. 12 is a current-voltage curve for perovskite solar cells according to Preparation Example 2 (experimental group) and Comparative Example 1 (control) of the present inventive concept;

FIG. 13 is a graph illustrating a change in the efficiency of solar cells over time, measured by a maximum power point tracking method when exposing the perovskite solar cells according to Preparation Example 2 (experimental group) and Comparative Example 1 (control) of the present inventive concept to light; and

FIG. 14 is a graph illustrating power conversion efficiency (PCE) values of perovskite solar cells of Comparative Example 1 and Preparation Examples 2 to 5 of the present inventive concept.

### DESCRIPTION OF EXAMPLE EMBODIMENTS

Hereinafter, example embodiments of the present inventive concept are disclosed with reference to the accompanying drawings.

The present inventive concept allows for various modifications and variations, as exemplified by the specific embodiments illustrated in the drawings, which will be described in detail below. However, it is not the intention to limit the scope of the invention to the disclosed particular forms. On the contrary, the invention encompasses all modifications, equivalents, and substitutions that are consistent with the principles and features of the invention as defined by the claims.

When elements such as layers, regions, or substrates are mentioned to exist “on” other components, it should be understood that they can either directly reside on the mentioned components or there may be intermediate elements between them. In other words, the presence of intermediate elements between the mentioned components is also possible.

It will be understood that, although the terms “first,” “second,” etc. may be used herein to describe various elements, components, areas, layers and/or regions, these elements, components, areas, layers and/or regions should not be limited by these terms.

### Phenanthroline-Based Compound

One aspect of the present inventive concept provides a phenanthroline-based compound represented by Formula 1 below.

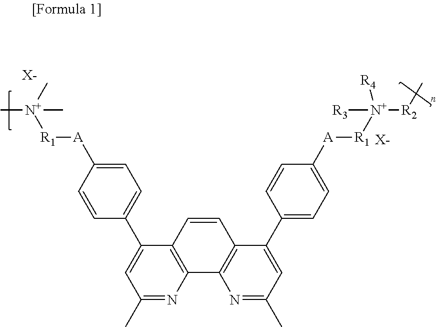

(In Formula 1, n is an integer of 1 to 1,000, R₁, R₂, R₃, and R₄ are C₁ to C₃₀ alkyls, A is oxygen, nitrogen, or a C₁ to C₁₀ alkyl, and X is a halogen.)

In Formula 1 representing the phenanthroline-based compound, each of Ri and R₂ may be hexyl, R₃ and R₄ may each be independently substituted or unsubstituted methyl, A may be oxygen, and X may be bromine.

The phenanthroline-based compound is a phenanthroline compound that includes phenanthroline with high electron affinity in the center and an ammonium halide group as a side chain, and is able to passivate defects in perovskite and metal oxide in a perovskite solar cell and effectively adjust energy levels of the underlying charge transport layer and the perovskite.

### Method of Preparing the Phenanthroline-Based Compound

In another aspect of the present inventive concept, a method of preparing the above-described phenanthroline-based compound is provided.

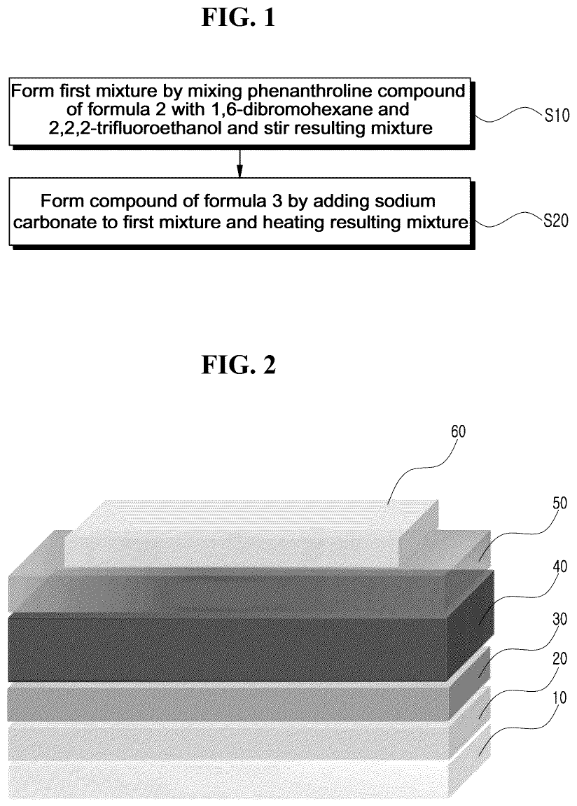

FIG. 1 is a flowchart showing a method of preparing a phenanthroline-based compound according to one embodiment of the present inventive concept.

Referring to FIG. 1, the method of preparing a phenanthroline-based compound may include forming a first mixture by mixing a phenanthroline compound of Formula 2 below with 1,6-dibromo hexane and 2,2,2-trifluoroethanol and stirring the resulting mixture (S10), and forming a compound of Formula 3 below by adding sodium carbonate to the first mixture and heating the resulting mixture (S20).

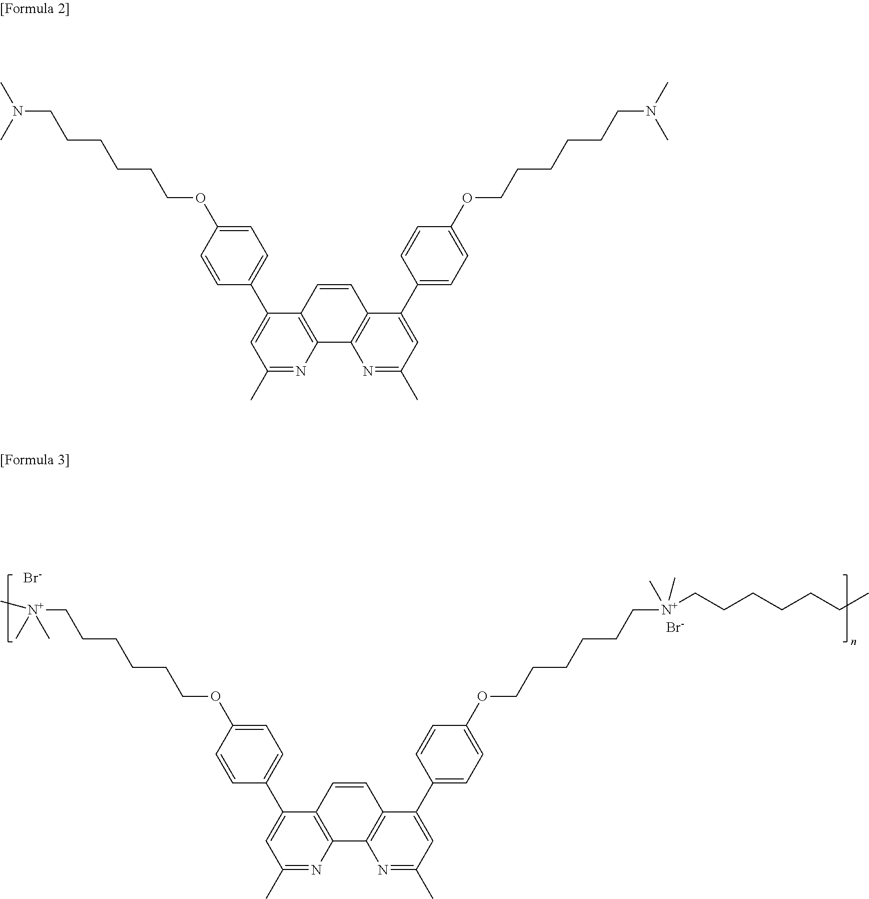

Scheme 1 below shows a reaction of synthesizing the phenanthroline compound of Formula 2 with 1,6-dibromo hexane in the presence of a solvent and an acidity regulator in S10 and S20.

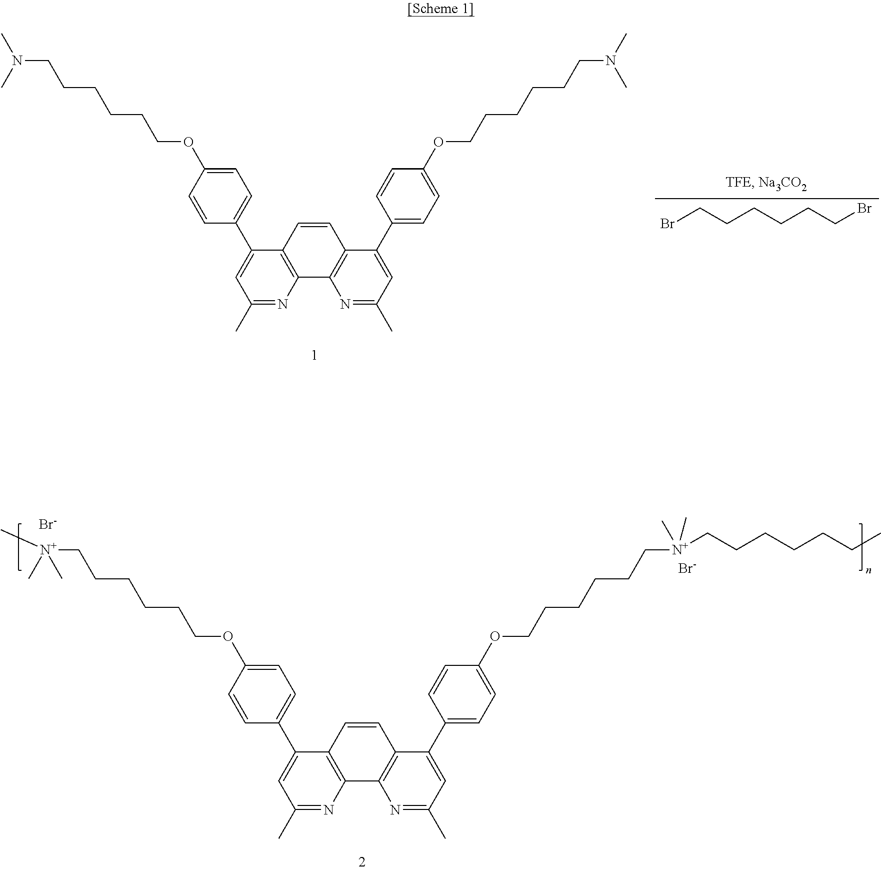

(In Scheme 1, n is defined as in Formula 1.)

First, a first mixture may be formed by mixing the phenanthroline compound of Formula 2,6,6′-{(2,9-dimethyl-1,10-phenanthroline-4,7-diyl)bis[(4,1-phenylene)oxy]}bis(N,N-dimethylhexane-1-amine), with 1,6-dibromo hexane and 2,2,2-trifluoroethanol, and stirring the resulting mixture at room temperature.

Subsequently, the compound of Formula 3 above may be formed by adding sodium carbonate to the first mixture and heating the resulting product. The compound of Formula 3 is poly [N-(6-{4-[2,9-dimethyl-7-(4-{[6-(trimethyl azaniumyl)hexyl]oxy}phenyl)-1,10-phenanthroline-4-yl]phenoxy}hexyl)-N,N-dimethylheptane-1-ammonium bromide].

Here, in the forming of the compound of Formula 3, heating may be performed at 70° C. to 80° C.

When the heating temperature for forming the compound of Formula 3 is less than 70° C., since the first mixture and the sodium carbonate do not sufficiently react, it may be difficult to form the target compound of Formula 3.

In addition, when the heating temperature for forming the compound of Formula 3 is more than 80° C., the process yield of the compound of Formula 3 may be lowered due to a side reaction.

After completing the synthesis reaction of the compound of Formula 3, adding hexane to the compound of Formula 3 and purifying the resulting product through a filter may be further included. Through the purification, the compound of Formula 3 may be formed with high purity.

In addition, the compound of Formula 2 may be formed by a process of forming a first mixture by mixing 4,7-bis[4-((6-bromohexyl)oxy)phenyl]-2,9-dimethyl-1,10-phenanthroline with tetrahydrofuran (THF), a process of forming a second mixture by mixing dimethylamine with the first mixture, a process of forming a third mixture by sequentially adding sodium hydroxide, ethyl acetate, and water to the second mixture, and a process of performing vacuum distillation by washing the third mixture with water.

That is, first, the compound of Formula 2 may be the first mixture, which is formed by dissolving the 4,7-bis[4-((6-bromohexyl)oxy)phenyl]-2,9-dimethyl-1,10-phenanthroline in THF as a solvent, and stirring the resulting mixture for 30 minutes to 40 minutes.

Here, the process of forming the first mixture may be performed at −80° C. to −70° C.

When the temperature during the process of forming the first mixture is lower than −80° C., due to the low temperature, the reactant may freeze without fully reacting.

In addition, when the temperature during the process of forming the first mixture is higher than −70° C., it may be difficult to obtain the first mixture at the desired amount due to a side reaction.

Next, a second mixture may be formed by adding dimethylamine to the first mixture and stirring the resulting product at room temperature until the end of the reaction.

Afterwards, after terminating the reaction by adding sodium hydroxide to the second mixture, a third mixture may be formed by sequentially adding ethyl acetate and water.

Subsequently, the compound of Formula 2 may be formed by washing the third mixture with water several times and then performing vacuum distillation.

The vacuum distillation is distillation performed under a low pressure, and may be performed using a conventional method and device.

In addition, purifying the compound of Formula 2 prepared after the vacuum distillation using column chromatography may be further included.

### Perovskite Solar Cells Including Phenanthroline-Based Compound

In still another aspect of the present inventive concept, a perovskite solar cell including a phenanthroline-based compound is provided.

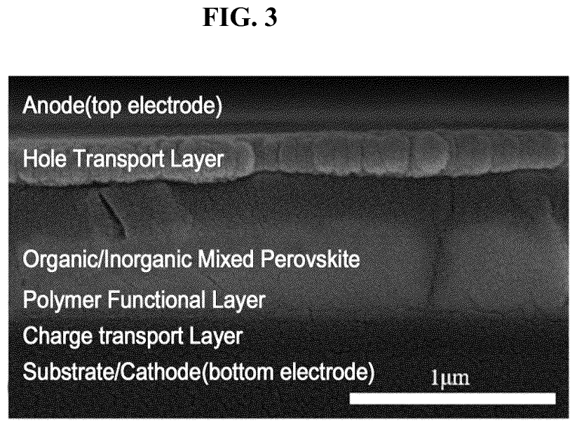

FIG. 2 is a schematic diagram illustrating a perovskite solar cell according to one embodiment of the present inventive concept, and FIG. 3 is an image illustrating the cross-section of a perovskite solar cell according to one embodiment of the present inventive concept.

Referring to FIGS. 2 and 3, the perovskite solar cell may include a bottom electrode (10), a charge transport layer (20) formed on the bottom electrode, a polymer functional layer (30) formed on the charge transport layer and including the phenanthroline-based compound of Formula 1 according to the present inventive concept, a perovskite photoactive layer (40) formed on the polymer functional layer, a hole transport layer (50) formed on the perovskite photoactive layer, and a top electrode (60) formed on the hole transport layer.

The bottom electrode (10) is a cathode into which an electron is injected and may consist of a material with conductivity. The bottom electrode (10) is preferably a conductive material such as a transparent metal, a metal oxide, or a carbon material. The metal may be silver nanowires, a metal thin film, or a combination of a metal thin film and a metal oxide. In addition, the metal oxide may be indium tin oxide (ITO), fluorine tin oxide (FTO), antimony tin oxide (ATO), fluorine-doped tin oxide (FTO), SnO₂, ZnO, or a combination thereof, and the carbon material may be graphite, graphene, carbon nanotubes, or a conductive polymer (PEDOT:PSS, polypyrrole, polyaniline, or poly thiophene).

The bottom electrode (10) may be formed on a substrate. The substrate may be a glass substrate, or maybe a sheet formed of a flexible material.

The bottom electrode (10) may be formed using physical vapor deposition (PVD), chemical vapor deposition (CVD), sputtering, pulse laser deposition (PLD), evaporation, electron-beam evaporation (E-beam evaporation), atomic layer deposition (ALD), or molecular beam epitaxial deposition (MBE).

The charge transport layer (20) may be formed on the bottom electrode (10). The charge transport layer (20) may serve to increase the injection or transfer efficiency of electrons into a perovskite thin film in the bottom electrode (10).

In the charge transport layer (20), as an n-type semiconductor, SnO₂, ZnO, TiO₂, a C₆₀ derivative, or [6,6]-phenyl-C61-butyric acid methyl ester (PCBM) may be included. Alternatively, the charge transport layer (20) may be diphenylphosphine oxide-4-(triphenylsilyl)phenyl (TSPO1), 1,3,5-tris (N-phenylbenzimidazol-2-yl)benzene (TPBi), tris(8-quinolinolate)aluminum (Alq3), a 2,5-diaryl silol derivative (PyPySPyPy), a perfluorinated compound (PF-6P), octa-substituted cyclooctatetraene (COTs), TAZ (refer to the following formula), 4,7-diphenyl-1,10-phenanthroline (Bphen), BCP (refer to the following formula), or BAlq (refer to the following formula).

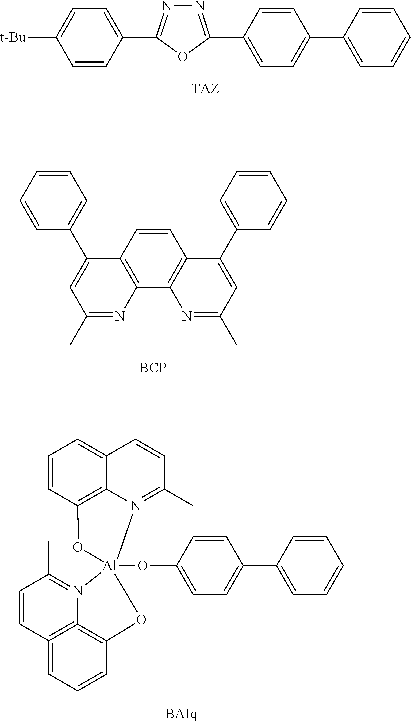

The charge transport layer (20) may be formed using spin coating, dip-coating, thermal deposition, or spray deposition.

The polymer functional layer (30) may be formed on the charge transport layer (20). The polymer functional layer (30) may include the phenanthroline-based compound of Formula 1 according to the present inventive concept. The above description of the phenanthroline-based compound may be referred to as the phenanthroline-based compound of Formula 1.

The phenanthroline-based compound of Formula 2 constituting the polymer functional layer (30) may serve as an electrolyte layer since a phenanthroline-based polymer material with an ammonium halide moiety is interposed between the charge transport layer (20) and the perovskite photoactive layer (40).

The polymer functional layer (30) may be introduced below the perovskite photoactive layer (40) to induce crystal growth in a vertical direction of perovskite crystals constituting the perovskite photoactive layer (40). The phenanthroline-based compound of the present inventive concept constituting the polymer functional layer (30) may have excellent processability, and thus may form a uniform thin film with low roughness even when formed as a solution process. Accordingly, the crystal growth of the perovskite may be induced in an appropriate direction, and defects in the perovskite photoactive layer may be minimized

The aforementioned polymer functional layer (30) may possess a passivation function. Specifically, the phenanthroline-based compound composing the polymer functional layer (30) contains phenanthroline with a high electron affinity in its central core while also having ammonium halide functionalities. This unique combination enables effective passivation (neutralization) of ionic defects in perovskite and metal oxides, thereby minimizing the loss of charge transport during solar cell operation.

When the perovskite solar cell is exposed to light and photostability is measured in a nitrogen atmosphere by a maximum power point tracking method, even after 750 hours, the solar cell's performance remains at 93% relative to the initial efficiency. That is, as the polymer functional layer (30) is disposed between the charge transport layer (20) and the perovskite layer (40), the energy levels of the charge transport layer (20) and the perovskite layer (40) may be adjusted, enabling efficient charge transfer. Accordingly, the perovskite solar cell of the present inventive concept may increase open-circuit voltage and photocurrent, enhancing power conversion efficiency and photostability. This will be described in detail based on the following preparation examples and experimental examples.

The polymer functional layer (30) may be disposed between the charge transport layer (20) and the perovskite layer (40), adjusting the energy levels of the charge transport layer (20) and the perovskite layer (40). This will be described in detail based on the following preparation examples and experimental examples.

The polymer functional layer (30) may have a thickness of 5 nm to 14 nm. When the thickness of the polymer functional layer (30) is less than 5 nm, because the polymer functional layer (30) is not uniformly formed, it does not easily perform its role as a functional layer and may have lower power conversion efficiency. In addition, when the thickness of the polymer functional layer (30) is more than 14 nm, the generated charges within the perovskite photoactive layer (40) may be hindered from reaching the charge transport layer (20) located beneath the polymer functional layer (30). As a result, the power conversion efficiency of the solar cell may be reduced.

The polymer functional layer (30) may have an RMS value of 0.18 nm to 0.2 nm. The phenanthroline-based compound of Formula 1 constituting the polymer functional layer (30) may have excellent processability and exhibit a lower RMS value than general-purpose BCP, and may not inhibit the crystal growth of the perovskite photoactive layer (40) formed on the polymer functional layer (30). Accordingly, the polymer functional layer (30) may form a uniform thin film by a solution process. This will be described in detail based on the following preparation examples and experimental examples.

The polymer functional layer (30) may be formed using bar coating, spray coating, slot die coating, gravure coating, blade coating, screen printing, nozzle printing, inkjet printing, electrohydrodynamic jet printing, electrospraying, or electrospinning

The perovskite photoactive layer (40) may be formed on the polymer functional layer (30). The perovskite photoactive layer (40) may include a perovskite material, which may be a material known to be suitable for a solar cell. For example, as the perovskite material, formamidinium lead bromide (FAPbBr₃), formamidinium lead bromine chloride (FAPbBr₂Cl), or methylammonium lead iodide (MAPbI₃) may be used, but the present inventive concept is not limited thereto.

Preferably, the perovskite photoactive layer (40) may be a triple cation-based halide perovskite, (CsPbI3)_{0.05}[(FAPbI₃)0.97(MAPbBr₃)_{0.03}]_{0.95}. FA is formamidinium and MA is methylammonium.

The perovskite photoactive layer (40) may be a bulk polycrystalline thin film or a thin film consisting of nanocrystal particles, and the nanocrystal particles may have a core-shell structure or a structure with gradient composition.

The perovskite photoactive layer (40) may be formed using bar coating, spray coating, slot die coating, gravure coating, blade coating, screen printing, nozzle printing, inkjet printing, electrohydrodynamic jet printing, electrospraying, or electrospinning

The hole transport layer (50) may be formed on the perovskite photoactive layer (40). The hole transport layer (50) may include, as a p-type semiconductor, a hole transport material conventionally used in the art.

General hole transport materials may include, for example, N,N-dicarbazolyl-3,5-benzene (mCP); poly (3,4-ethylenedioxythiophene):polystyrenesulfonate (PEDOT:PSS); N,N′-di(1-naphthyl)-N,N′-diphenylbenzidine (NPD); N,N′-diphenyl-N,N′-di(3-methylphenyl)-4,4′-diaminobiphenyl (TPD); N,N′-diphenyl-N,N′-dinaphthyl-4,4′-diaminobiphenyl; N,N,N′N′-tetra-p-tolyl-4,4′-diaminobiphenyl; N,N,N′N′-tetraphenyl-4,4′-diaminobiphenyl; a porphyrin compound derivative such as copper(II)1,10,15,20-tetraphenyl-21H,23H-porphyrin; 1,1-bis[4-[N,N′-di(p-tolyl)amino]phenyl]cyclohexane (TAPC); a triarylamine derivative such as N,N,N-tri(p-tolyl) amine, 4,4′,4′-tris[N-(3-methylphenyl)-N-phenylamino]triphenylamine; carbazole derivatives such as N-phenylcarbazole and polyvinylcarbazole; phthalocyanine derivatives such as non-metal phthalocyanines and copper phthalocyanine; starburst amine derivatives; N-aminostilbene-based derivatives; derivatives of aromatic tertiary amines and styryl amine compounds; and polysilane. Specifically, the hole transport layer (50) may include Spiro-OMeTAD, MoOx, or PEDOT:PSS.

Preferably, the hole transport layer (50) may include 2,2′,7,7′-tetrakis[N,N-di(4-methoxyphenyl)amino]-9,9′-spirobifluorene (Spiro-OMeTAD).

The hole transport layer (50) may be formed using spin coating, dip coating, thermal deposition or spray deposition.

The top electrode (60) may be formed on the hole transport layer (50).

The top electrode (60) may be a conductive metal oxide, a metal, a metal alloy, or a carbon material. The conductive metal oxide may be indium tin oxide (ITO), fluorine tin oxide (FTO), antimony tin oxide (ATO), FTO, SnO₂, ZnO, or a combination thereof. A metal or metal alloy suitable for the top electrode (60) may be gold (Au) or copper iodine (CO. The carbon material may be graphite, graphene, or carbon nanotubes.

The top electrode (60) may be formed using physical vapor deposition (PVD), chemical vapor deposition (CVD), sputtering, pulse laser deposition (PLD), evaporation, electron-beam evaporation E-beam evaporation, atomic layer deposition (ALD), or molecular beam epitaxial deposition (MBE).

Hereinafter, preferred preparation examples and experimental examples will be presented to better explain the present inventive concept. However, the following preparation examples and experimental examples are merely provided to better explain the present inventive concept, and the present inventive concept is not limited to the following preparation examples.

### Preparation Example 1

### Preparation of Phenanthroline-Based Compound

As shown in Scheme 1, a novel electrolyte polymer material, poly[N-(6-{4-[2,9-dimethyl-7-(4-{[6-(trimethylazaniumyl)hexyl]oxy}phenyl)-1,10-phenanthroline-4-yl]phenoxy}hexyl)-N,N-dimethylheptane-1-ammonium bromide], is prepared by the polymerization of 6,6′-{(2,9-dimethyl-1,10-phenanthroline-4,7-diyl)bis[(4,1-phenylene)oxy]}bis (N,N-dimethylhexane-1-amine) and 1,6-dibromo hexane.

(1) Preparation of 6,6′-{(2,9-dimethyl-1,10-phenanthroline-4,7-diyl)bis[(4,1-phenylene)oxy]}bis(N,N-dimethylhexane-1-amine)

4,7-bis[4-((6-bromohexyl)oxy)phenyl]-2,9-dimethyl-1,10-phenanthroline (4.5 g, 6.26 mmol) is dissolved in tetrahydrofuran (80 mL), and stirred at −78° C. for 30 minutes.

Subsequently, dimethylamine (62.6 mL, 125.3 mmol) is added, and stirring is performed at room temperature until the reaction is terminated.

And then, 0.1 M sodium hydroxide is added to terminate the reaction, and ethyl acetate and water are added.

The reacted mixture is washed with 100 mL of water three times and subjected to vacuum distillation, obtaining a yellow oil as a residue. The oil is separated through column chromatography, thereby obtaining the compound of Formula 2 in the form of a light-yellow solid (2.5 g, 62%).

¹H NMR (300 MHz, CDCl3): δ (ppm) 7.797 (s, 2H) 7.455 (d, 4H, J=8.9 Hz) 7.422 (s, 2H) 7.040 (d, 4H, J=8.8 Hz) 4.050 (t, 4H, J=6.5 Hz) 2.979 (s, 6H) 2.312 (t, 4H, J=7.0 Hz) 2.249 (s, 12H) 1.92-1.80 (m, 4H) 1.60-1.48 (m, 8H), 1.48-1.38 (m, 4H);

¹³C NMR (75 MHz, CDCl3): δ (ppm) 159.263, 158.543, 148.249, 146.028, 130.909, 130.342, 124.782, 123.878, 122.882, 114.503, 68.028, 59.771, 45.464, 29.226, 27.649, 27.266, 26.071, 25.902.

Estimated HRMS (m/z, Er) value, 646.4250, and measured HRMS (m/z, Er) value, 646.4247, for C₄2H₅₄N₄O₂

(2) Preparation of poly[N-(6-{4-[2,9-dimethyl-7-(4-{[6-(trimethylazaniumyl)hexyl]oxy}phenyl)-1,10-phenanthroline-4-yl]phenoxy}hexyl)-N,N-dimethylheptane-1-ammonium bromide]

The 6,6′-{(2,9-dimethyl-1,10-phenanthroline-4,7-diyl)bis[(4,1-phenylene)oxy]}bis(N,N-dimethylhexane-1-amine) (0.1 g, 0.1546 mmol) prepared in (1) and 1,6-dibromo hexane (0.02 mL, 0.1546 mmol) are dissolved in 2,2,2-trifluoroethanol (3 mL), and stirred at room temperature.

Afterwards, sodium carbonate (0.033 mL, 0.3138 mmol) is added and heated at 70° C.

After the end of the reaction, hexane is added, and a yellow solid (0.4 g, 27%) is obtained as a residue through filtration.

GPC (Mn=10.2 kDa). ¹H NMR (300 MHz, CDCl3): δ (ppm) 7.756 (s, 1H) 7.460-7.380 (m, 3H) 7.017 (d, 2H, J=8.8 Hz) 4.601-4.250 (m, 2H) 4.076 (t, 2H, J=5.8 Hz) 3.620-3.500 (m, 4H) 3.480-3.400 (m, 2H) 3.423 (s, 6H) 3.380-3.200 (m, 4H) 2.971 (s, 3H) 1.850-1.250 (m, 22H)

### Preparation Example 2

### Preparation of Perovskite Solar Cell Including Phenanthroline-Based Compound of Preparation Example 1 as Polymer Functional Layer

A perovskite solar cell having the structure shown in FIG. 2 is prepared.

As a bottom electrode (10), a transparent electrode (ITO) is formed to a thickness of 150 nm on a glass substrate using vacuum deposition.

To prepare a charge transport layer (20), 15 wt % of tin oxide nanoparticles (Alfa Aesar) is diluted in distilled water at a concentration of 2.5 wt %, and then coated on the bottom electrode (10) by spin coating at 3,000 rpm for 30 seconds. After completing the spin coating, the substrate is subjected to a drying process for 30 minutes on a hot plate maintained at 150° C., thereby forming the charge transport layer (20).

A polymer functional layer (30) is formed to a thickness of 9 nm by preparing a solution in which the phenanthroline-based compound prepared in Preparation Example 1 is diluted in methanol at 1.0 mg/mL, and coating the charge transport layer 20 with the prepared solution by spin coating at 5,000 rpm for 30 seconds.

To prepare a perovskite photoactive layer (40), a solution prepared by dissolving PbI₂, PbBr₂, CsI, FAI, MAI and MAC1 in dimethylformamide (DMF)/dimethyl sulfoxide (DMSO) (4:1) at a ratio of 1.5 mol (CsPbI₃)_{0.05}[(FAPbI₃)_{0.97}(MAPbBr₃)_{0.03}]_{0.95}+30 mol % MAC1 is applied on the surface coated with the polymer functional layer (30), and a perovskite thin film is formed using first spin coating at 1,000 rpm for 5 seconds and second spin coating at 5,000 rpm for seconds. To form crystals of the perovskite thin film, during the second spin coating of the perovskite solution, a process of spraying a diethyl ether (DEE) solution 5 seconds before the end of the spin coating is performed. After spin coating, the substrate on which the perovskite thin film is formed is dried on a hot plate maintained at 150° C. for 10 minutes.

2,2′,7,7′-tetrakis (N,N-di-4-methoxyphenylamine)-9,9′-spirobifluorene(Spiro-OMeTAD) used as a hole transport layer (50) is dissolved in a chlorobenzene(Aldrich) solvent at the rate of 91 mg/mL. Doping of the hole transport layer solution is carried out by mixing said solution with a solution of bis(trifluoromethane) sulfonimide lithium salt (Li-TFSI) dissolved in 21.02 uL of acetonitrile(Aldrich) solution at a ratio of 520 mg/ml and a solution of tris(2-(1H-pyrazol-1-yl)-4-tert-butylpyridine)-cobalt(III) triThis(trifluoromethane)sulfonimidel(FK209) dissolved in 10 uL of acetonitrile solution at a ratio of 375 mg/ml. The perovskite photoactive layer (40) is coated with spiro-OMeTAD using a spin-coating method at 3000 rpm for 30 seconds to form a doped spiro-OMeTAD thin film.

As a top electrode (60), Au is formed to a thickness of 100 nm on the hole transport layer (50) by vacuum deposition, thereby producing a perovskite solar cell.

### Preparation Example 3

### Production of Perovskite Solar Cell Using Polymer Functional Layer with Different Thickness

A perovskite solar cell is prepared in the same manner as in Preparation Example 2, except that a polymer functional layer is formed to a thickness of 3 nm.

### Preparation Example 4

### Production of Perovskite Solar Cell Using Polymer Functional Layer with Different Thickness

A perovskite solar cell is prepared in the same manner as in Preparation Example 2, except that a polymer functional layer is formed to a thickness of 5 nm.

### Preparation Example 5

### Production of Perovskite Solar Cell Using Polymer Functional Layer with Different Thickness

A perovskite solar cell is prepared in the same manner as in Preparation Example 2, except that a polymer functional layer is formed to a thickness of 14 nm.

### Comparative Example 1

### Production of Perovskite Solar Cell Without Phenanthroline-Based Compound of Preparation Example 1 as Polymer Functional Layer

A perovskite solar cell is prepared in the same manner as in Preparation Example 2, excluding the process of forming a polymer functional layer.

### Experimental Example 1

### Measurement of the Roughness of Film Consisting of Existing BCP Film and Phenanthroline-Based Compound of Formula 1 of Present Inventive Concept

To compare the characteristics of existing general-purpose BCP and the phenanthroline-based compound of Formula 1 according to the present inventive concept, a film is formed of each material, and an RMS indicating the overall roughness of the film is measured. The film is prepared in the same manner as described above using a conventional solution process method.

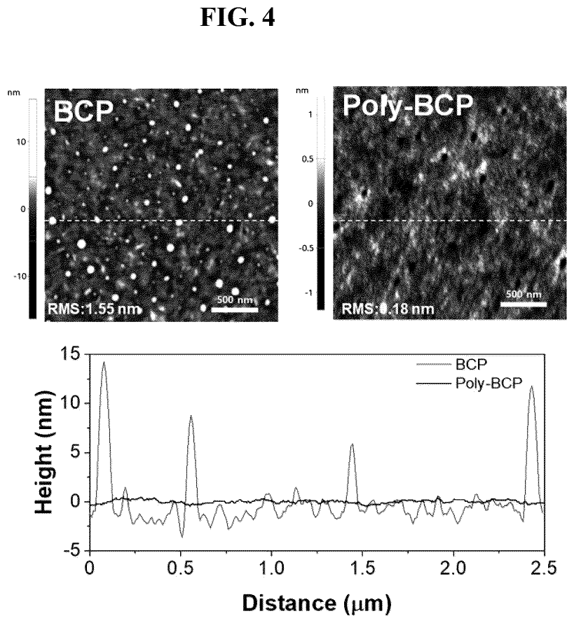

FIG. 4 is a set of images and a graph, which show the result of RMS measurement on the surfaces of a film consisting of a phenanthroline-based compound of Preparation Example 1 of the present inventive concept and a film consisting of general-purpose BCP.

Referring to FIG. 4, when comparing the roughnesses of the BCP film and the film consisting of Preparation Example 1, it can be confirmed that the BCP film is non-uniformly coated in a dotted form in a solution process and have a high RMS, which is a measure of roughness, of 1.55 nm. This is due to the fact that BCP is a small-molecule material.

On the other hand, it can be seen that the film consisting of the phenanthroline-based compound of Preparation Example 1 of the present inventive concept has a uniform thickness profile of several nanometers or less, and its RMS may be 0.18 nm, which is significantly lower than that of the BCP film.

As shown in the graph of FIG. 4, while the BCP film has a great height difference, it can be confirmed that the film formed as Preparation Example 1 (shown in red) has almost no height difference. This can be seen as a result of the improved processability of the phenanthroline-based compound of Preparation Example 1 of the present inventive concept.

As described above, the phenanthroline-based compound of the present inventive concept forms a uniform thin film with low roughness even when formed by a solution process, and may lead a perovskite grain to grow in the right direction when introduced as a polymer functional layer between a charge transport layer and a perovskite photoactive layer in a perovskite solar cell.

### Experimental Example 2

### Measurement of Crystallinity of Perovskite Photoactive Layer Depending on Formation of Polymer Functional Layer

For the perovskite solar cell according to the present inventive concept, an experiment is carried out to examine the change in crystallinity of the perovskite photoactive layer in the perovskite solar cell depending on the formation of a polymer functional layer consisting of the compound of Formula 1 on a charge transport layer.

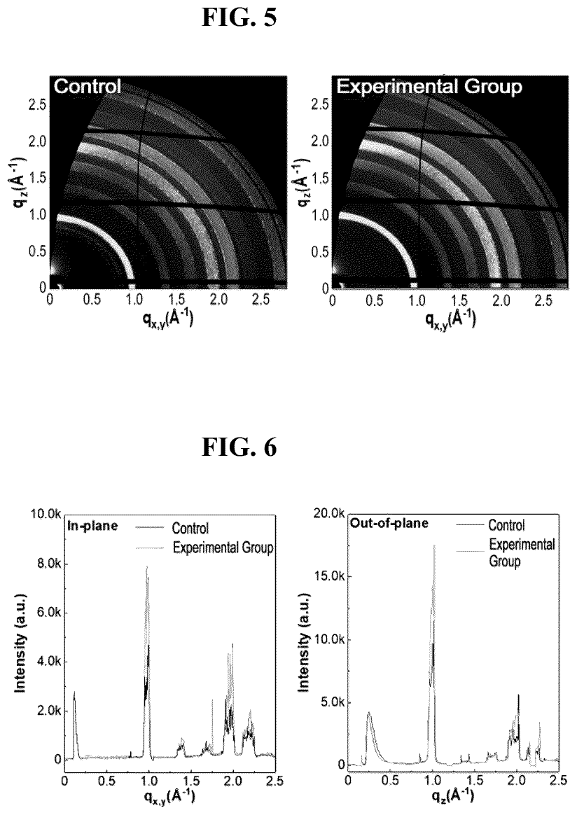

2D grazing-incidence X-ray diffraction (GIXRD) is performed on the perovskite photoactive layers of Preparation Example 2 and Comparative Example 1, and 1-D extraction and azimuths are measured and are shown in FIGS. 5 to FIGS. 7. Conventional methods are used for each measurement method.

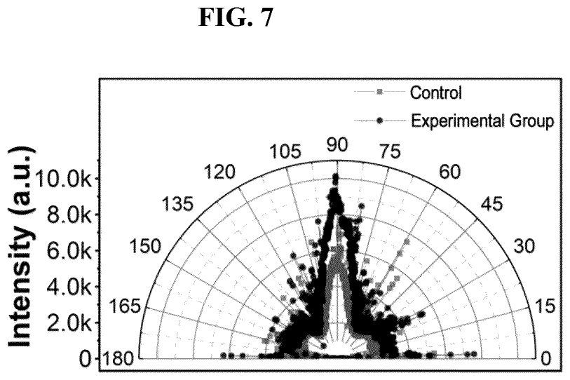

FIG. 5 is a set of images of measuring 2D grazing-incidence X-ray diffraction (GIXRD) of perovskite photoactive layers in perovskite solar cells according to Preparation Example 2 and Comparative Example 1 of the present inventive concept, FIG. 6 is a set of graphs illustrating 1-D extraction results for perovskite photoactive layers in perovskite solar cells according to Preparation Example 2 and Comparative Example 1 of the present inventive concept, and FIG. 7 is an azimuthal diagram of perovskite photoactive layers in perovskite solar cells according to Preparation Example 2 and Comparative Example 1 of the present inventive concept.

Referring to FIGS. 5 to FIGS. 7, compared to Comparative Example 1 (control), it can be confirmed that the crystallinity of the perovskite material constituting the perovskite photoactive layer of Preparation Example 2 (experimental group) increased. Particularly, it can be seen that the growth of the perovskite material in a vertical direction is further improved.

### Experimental Example 3

### Measurement of the Work Function of Charge Transport Layer Depending on Formation of Polymer Functional Layer

According to the present inventive concept, an experiment is carried out for the perovskite solar cell to examine the change in work function of a charge transport layer depending on the formation of the polymer functional layer consisting of the compound of Formula 1 on the charge transport layer.

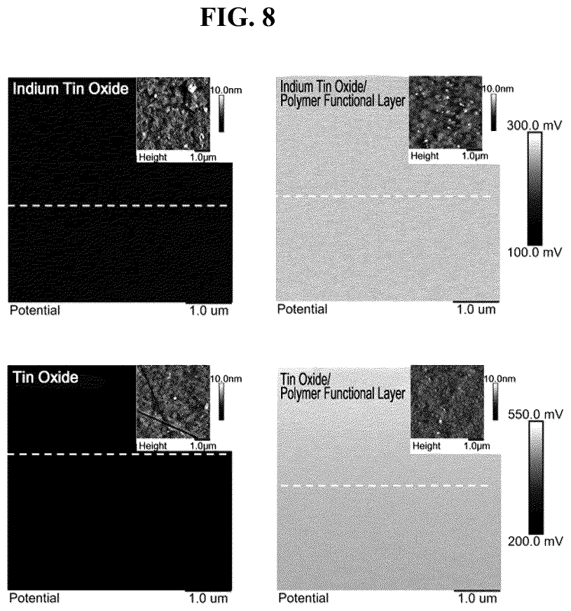

FIG. 8 is a set of images observing charge transport layers in perovskite solar cells according to Preparation Example 2 and Comparative Example 1 of the present inventive concept using a Kelvin probe force microscope (KPFM). Referring to FIG. 8, it can be confirmed that the charge transport layer of

Preparation Example 2 including the polymer functional layer of the present inventive concept has a lower roughness and a more uniform profile, compared to the charge transport layer of Comparative Example 1.

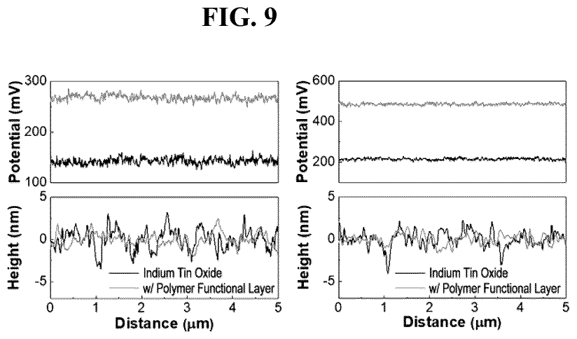

FIG. 9 is a set of graphs illustrating the result of measuring 1-D(one-dimension) extracted from the images observed by a Kelvin probe atomic force microscope on charge transport layers in perovskite solar cells according to Preparation Example 2 and Comparative Example 1 of the present inventive concept.

Referring to FIG. 9, it can be confirmed that, in Preparation Example 2 (shown in red) including the polymer functional layer of the present inventive concept, the work function of the charge transport layer formed of a metal oxide shifts toward the side of n-type. Accordingly, it can be seen that the charge transfer of the charge transport layer in the perovskite solar cell of Preparation Example 2 including the polymer functional layer of the present inventive concept is more efficient compared to Comparative Example 1.

### Experimental Example 4

### Detection of Defect Passivation of Charge Transport Layer Depending on Formation of Polymer Functional Layer

According to the present inventive concept, an experiment is carried out for the perovskite solar cell to detect the defect passivation of a charge transport layer depending on the formation of the polymer functional layer consisting of the compound of Formula 1 on the charge transport layer.

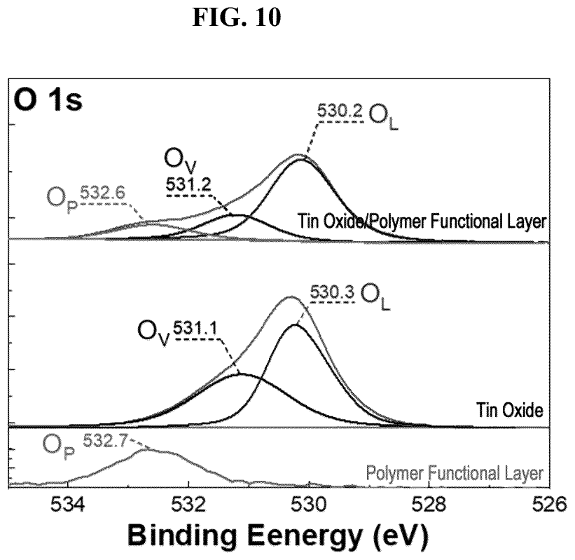

FIG. 10 is a graph for oxygen elements in data obtained by X-ray photoelectron spectroscopy (XPS) of the charge transport layers in perovskite solar cells according to Preparation Example 2 and Comparative Example 1 of the present inventive concept.

Referring to FIG. 10, it can be confirmed that oxygen vacancy, which is a surface defect of the metal oxide charge transport layer made by a low-temperature solution process, is passivated. Accordingly, as a polymer functional layer consisting of a phenanthroline-based compound is introduced between a charge transport layer and a perovskite photoactive layer in the perovskite solar cell of the present inventive concept, the defect of the charge transport layer formed by the low-temperature solution process is passivated, and the power conversion efficiency and photostability of the solar cell may be improved through smooth charge transfer.

### Experimental Example 5

### Measurement of Defect Density of Perovskite Photoactive Layer Depending on Formation of Polymer Functional Layer

According to the present inventive concept, an experiment is carried out for the perovskite solar cell to measure the defect density of the perovskite photoactive layer depending on the formation of the polymer functional layer consisting of the compound of Formula 1 on the charge transport layer.

As a defect calculation formula, the following Equation (1) is used.

nₜ=2ϵ₀ϵᵣV_{TFL}/qL² Equation (1)

(In Equation (1), nₜ is a defect concentration, ϵ₀ is a vacuum permittivity, ϵᵣ is the relative permittivity of the perovskite, V_{TFL} is a trap-filled limit voltage, q is an elementary charge, and L is a film thickness.)

As a mobility calculation formula, the following Equation (2) is used.

$$\\ \begin{matrix}J = \frac{9}{8} {ε}_{0} {ε}_{r} μ \frac{{V}^{2}}{{L}^{3}} & Equation ( 2 )\end{matrix}$$

(In Equation (2), μ is mobility, [symbol] is a current, and V is a voltage.)

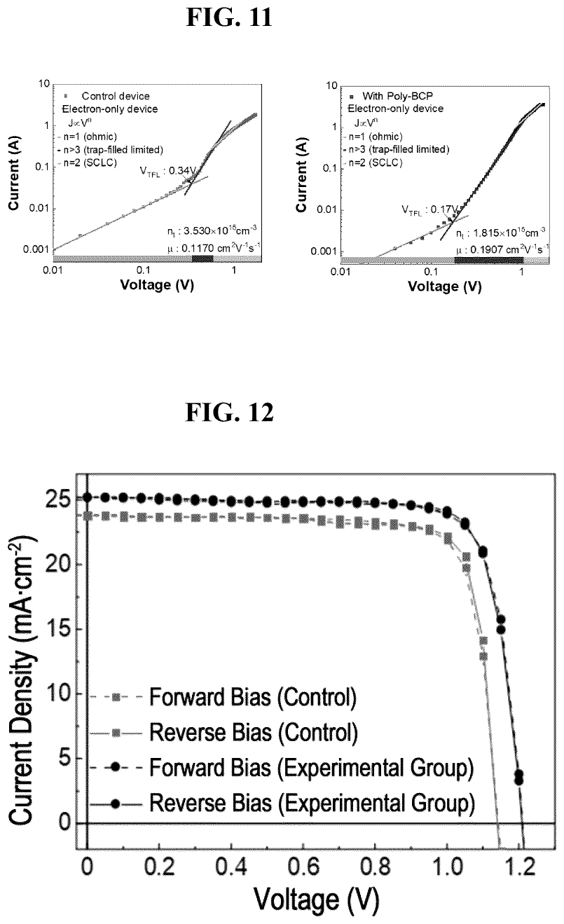

FIG. 11 is a set of graphs illustrating space charge limited current curves for the perovskite photoactive layers in perovskite solar cells according to Preparation Example 2 and Comparative Example 1 of the present inventive concept.

Referring to FIG. 11, it can be confirmed that the density of defects of the perovskite material causing the performance loss of the perovskite solar cell is reduced. In this way, in the perovskite solar cell of the present inventive concept, by introducing a polymer functional layer consisting of the phenanthroline-based compound between a charge transport layer and a perovskite photoactive layer, the density of ionic defects formed during the production of the solar cell may be reduced, further improving the optical and electrical properties of the perovskite material.

### Experimental Example 6

### Measurement of Performance of Perovskite Solar Cell Depending on Formation of Polymer Functional Layer

For the perovskite solar cell according to the present inventive concept, an experiment is carried out to measure the performance of the perovskite solar cell depending on the formation of the polymer functional layer consisting of the compound of Formula 1 on the charge transport layer.

FIG. 12 is a current-voltage curve for perovskite solar cells according to Preparation Example 2 (experimental group) and Comparative Example 1 (control) of the present inventive concept.

Referring to FIG. 12, it can be confirmed that the performance of the solar cell of Preparation Example 2 including the polymer functional layer is improved compared to Comparative Example 1. This can be seen as a result of passivating the ionic defects of the charge transport layer and the perovskite photoactive layer by the introduction of the polymer functional layer consisting of the phenanthroline-based compound, and facilitating charge transfer.

### Experimental Example 7

### Measurement of Photostability of Perovskite Solar Cell Depending on Formation of Polymer Functional Layer

When the perovskite solar cell is exposed to light, ionic defects in the perovskite layer move to each electrode, resulting in destruction of the electrode and an n-type functional layer. At the same time, the site where the ionic defects move upward induces defects in perovskite crystals, inhibiting the stability of the perovskite layer.

For the perovskite solar cell according to the present inventive concept, an experiment is carried out to measure the photostability of the perovskite solar cell depending on the formation of the polymer functional layer consisting of the compound of Formula 1 on the charge transport layer.

Specifically, the perovskite solar cells of Preparation Example 2 and Comparative Example 1 are exposed to sunlight, and solar cell efficiency over light exposure time is measured under a nitrogen atmosphere by maximum power point tracking method.

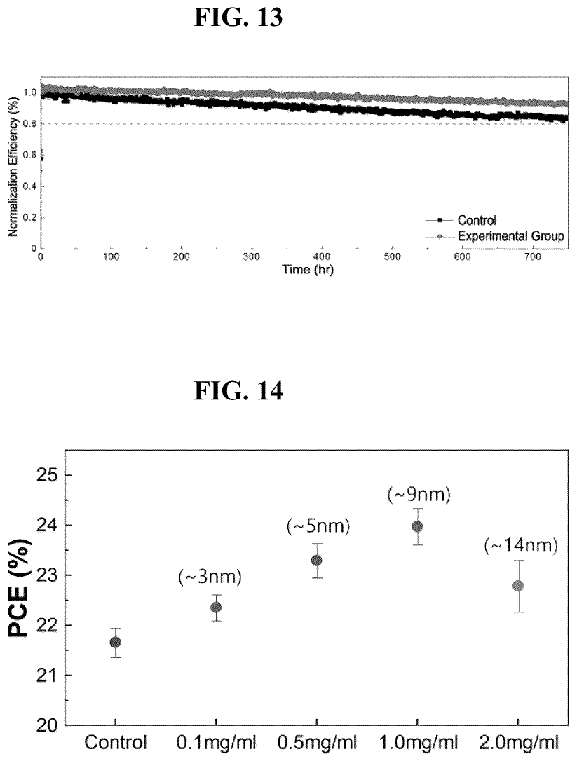

FIG. 13 is a graph illustrating a change in the efficiency of solar cells over time, measured by a maximum power point tracking method when exposing the perovskite solar cells according to Preparation Example 2 (experimental group) and Comparative Example 1 (control) of the present inventive concept to light.

Referring to FIG. 13, it can be confirmed that, in the perovskite solar cell of Preparation Example 2, even after 750 hours, the performance of the solar cell is maintained within 93% of the initial efficiency. On the other hand, it can be confirmed that, in the perovskite solar cell of Comparative Example 1, at the same time, the performance of the solar cell gradually decreases to 83% compared to the initial efficiency.

In this way, in the perovskite solar cell of the present inventive concept, photostability may be enhanced by the polymer functional layer consisting of the phenanthroline-based compound, effectively improving the performance of the solar cell.

### Experimental Example 8

### Measurement of Power Conversion Efficiency (PCE) of Perovskite Solar Cell According to Thickness of Polymer Functional Layer

For the perovskite solar cell according to the present inventive concept, an experiment is carried out to measure the PCE of the perovskite solar cell according to the thickness of the polymer functional layer consisting of the compound of Formula 1.

For the experiment, the perovskite solar cells are produced with different thicknesses of the polymer functional layers according to Preparation Examples 2 to 5.

FIG. 14 is a graph illustrating PCE values of perovskite solar cells of Comparative Example 1 and Preparation Examples 2 to 5 of the present inventive concept.

Referring to FIG. 14, it can be confirmed that the PCE of a perovskite solar cell which includes a polymer functional layer with a thickness of 5 nm to 14 nm is high at 22.5% or more. Particularly, the perovskite solar cell of Preparation Example 2, in which the thickness of a polymer functional layer is 9 nm exhibited the highest PCE value of approximately 25%.

As described above, in the perovskite solar cell of the present inventive concept, the polymer functional layer disposed between the charge transport layer and the perovskite photoactive layer may be formed to have a thickness in an appropriate range, facilitating charge transfer and thus improving the PCE of the solar cell.

A phenanthroline-based compound of Formula 1 of the present inventive concept can allow perovskite crystals to grow in a vertical direction through introduction below a perovskite photoactive layer as a polymer functional layer.

In addition, the polymer functional layer including the phenanthroline-based compound of the present inventive concept may serve as a passivation layer that passivates the ionic defects on the both surface of metal oxide and a perovskite material, formed by a low-temperature solution process.

In addition, the polymer functional layer including the phenanthroline-based compound of the present inventive concept may enable smooth charge transfer between two layers through a change in energy level by being introduced between the charge transport layer and the perovskite photoactive layer.

Moreover, the polymer functional layer including the phenanthroline-based compound of the present inventive concept may enhance the power conversion efficiency and photostability of a perovskite solar cell by simple functional layer introduction without the modification of a photoactive layer.

Technical effects of the present inventive concept are not limited to those mentioned above, and other technical effects not mentioned will be clearly understood by those of ordinary skill in the art from the description below.

The above description of the present inventive concept is merely provided to exemplify the present inventive concept, and it will be understood by those of ordinary skill in the art to which the present inventive concept belongs that the present inventive concept can be implemented in modified forms without departing from the essential features of the present inventive concept. Therefore, the exemplary embodiments described above should be interpreted as illustrative and not limited in any aspect. For example, each component described as a single unit may be implemented in a distributed manner, and components described as being distributed may also be implemented in a combined form.

The scope of the present inventive concept is represented by the following claims, and all changes or modifications derived from the meaning and scope of the claims and their equivalent concepts should be interpreted as being included in the scope of the present inventive concept.

What is claimed is:

1. A phenanthroline-based compound is represented by Formula 1 below. 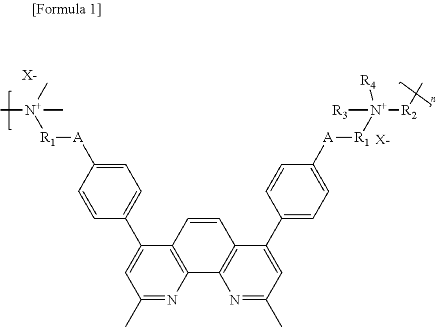
(In Formula 1, n is an integer of 1 to 1,000, R₁, R₂, R₃, and R₄ are C₁ to C₃₀ alkyls, A is oxygen, nitrogen, or a C₁ to C₁₀ alkyl, and X is a halogen.)

2. The compound of claim 1, wherein,
in Formula 1,
each of R₁ and R₂ is hexyl,
R₃ and R₄ are each independently substituted or unsubstituted methyl,
A is oxygen, and
X is bromine.

3. A method of preparing the phenanthroline-based compound of claim 1, comprising:
forming a first mixture by mixing a phenanthroline compound of Formula 2 below with 1,6-dibromo hexane and 2,2,2-trifluoroethanol and stirring the resulting mixture; and
forming a compound of Formula 3 below by adding sodium carbonate to the first mixture and heating the resulting mixture.

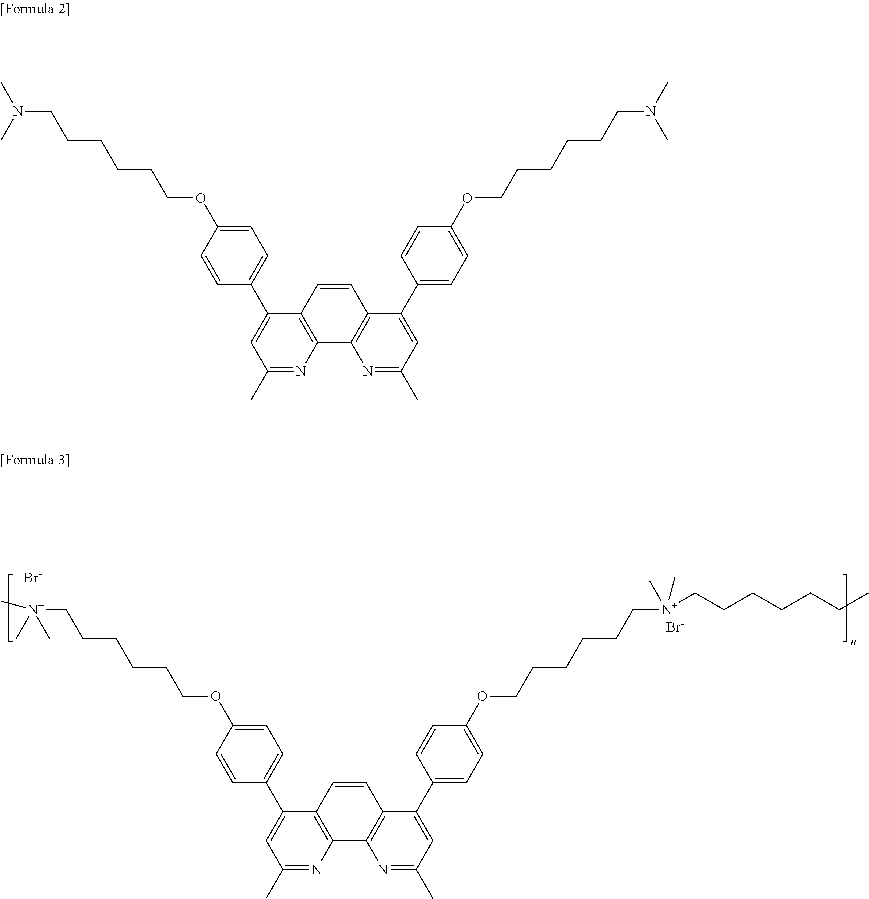

4. The method of claim 3, wherein the forming of the compound of Formula 3 comprises heating at 70° C. to 80° C.

5. The method of claim 3, wherein the compound of Formula 2 is formed by a process of forming a first mixture by mixing 4,7-bis [4-((6-bromohexyl)oxy)phenyl]-2,9-dimethyl-1,10-phenanthroline with tetrahydrofuran, a process of forming a second mixture by mixing dimethylamine with the first mixture, a process of forming a third mixture by sequentially adding sodium hydroxide, ethyl acetate, and water to the second mixture, and a process of performing vacuum distillation by washing the third mixture with water.

6. The method of claim 5, wherein the forming of the first mixture is performed at −80° C. to −70° C.

7. A perovskite solar cell, comprising:
a bottom electrode;
a charge transport layer formed on the bottom electrode;
a polymer functional layer formed on the charge transport layer, and comprising a phenanthroline-based compound of Formula 1 below;
a perovskite photoactive layer formed on the polymer functional layer;
a hole transport layer formed on the perovskite photoactive layer; and
a top electrode formed on the hole transport layer. 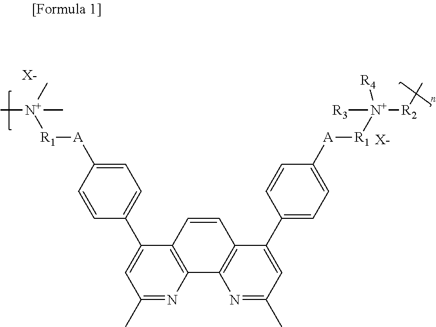
(In Formula 1, n is an integer of 1 to 1,000, R₁, R₂, R₃, and R₄ are C₁ to C₃₀ alkyls, A is oxygen, nitrogen, or a C₁ to C₁₀ alkyl, and X is a halogen.)

8. The perovskite solar cell of claim 7, wherein the polymer functional layer has a passivation function.

9. The perovskite solar cell of claim 7, wherein the polymer functional layer is formed to a thickness of 5 nm to 14 nm.

10. The perovskite solar cell of claim 7, wherein the polymer functional layer has an average surface roughness (root mean square, RMS) value of 0.18 nm to 0.2 nm.

11. The perovskite solar cell of claim 7, wherein, when the perovskite solar cell is exposed to light and photostability is measured in a nitrogen atmosphere by a maximum power point tracking method, even after 750 hours, the solar cell's performance remains at 93% relative to the initial efficiency.
